(19) United States
(12) Patent Application Publication
(10) Pub. No.: US 2025/0127938 A1
(43) Pub. Date: Apr. 24, 2025

(54) THERAGNOSTIC FOLATE CONJUGATES

(71) Applicant: Merck Patent GmbH, Darmstadt (DE)

(72) Inventors: Viola GROEHN, Altdorf / Uri (CH); Marie-Luise RENZ, Altdorf / Uri (CH); Rudolf MOSER, Altdorf / Uri (CH); Roger SCHIBLI, Villigen (CH); Cristina Magdalena MUELLER, Villigen (CH); Luisa Maria DEBERLE, Villigen (CH); Anna Elisabeth BECKER, Villigen (CH); Sara Dorina BUSSLINGER, Villigen (CH)

(73) Assignee: Merck Patent GmbH, Darmstadt (DE)

(21) Appl. No.: 18/834,636

(22) PCT Filed: Jan. 30, 2023
(86) PCT No.: PCT/EP2023/052117

§ 371 Date: Jul. 31, 2024

(30) Foreign Application Priority Data
Feb. 1, 2022 (EP) 22154652.6

(51) Int. Cl. A61K 51/04; A61P 35/00

(52) U.S. Cl. CPC A61K 51/0497; A61K 51/0459; A61P 35/00; A61K 2121/00; A61K 2123/00

(57) ABSTRACT

The present disclosure relates to new folate-conjugates comprising a 5-methyltetrahydro-folate, a radiometal chelator optionally coordinating a radiometal M, and the albumin binder 5-(p-iodophenyl)pentanoate, and further provides uses of such conjugates and/or pharmaceutical compositions thereof in diagnostic imaging, radionuclide therapy or theragnostic applications.

### FIELD OF THE DISCLOSURE

The present disclosure relates to new folate-conjugates comprising a 5-methyltetrahydrofolate, a radiometal chelator optionally coordinating a radiometal M, and the albumin binder 5-(p-iodophenyl)pentanoate, and further provides uses of such conjugates and/or pharmaceutical compositions thereof in diagnostic imaging, radionuclide therapy or theragnostic applications.

### BACKGROUND OF THE DISCLOSURE

The folate receptor-alpha (herein referred to as FR) is a membrane-associated glycoprotein which is overexpressed on a variety of tumor types, among those ovarian, lung, breast, renal and colorectal cancer (Parker, N. et al, Anal Bioch 2005, 338, (2), 284-93; Low, P. S. et al., Curr Opin Chem Biol 2009, 13, (3), 256-62). In healthy tissues, the FR is expressed at only a few sites, among them most importantly in the proximal tubule cells of the kidneys (Holm, J. et al., Kidney Int 1992, 41, (1), 50-5; Birn, H. et. al., J Am Soc Nephrol: JASN 2005, 16, (3), 608-15). It is, thus, a promising tumor-associated protein for nuclear imaging and targeted radionuclide therapy (Low, P. S. et al., Acc Chem Res 2008, 41, (1), 120-9; Müller, C. Curr Pharm Design 2012, 18, (8), 1058-83). Folic acid (oxidized version of folate vitamins) as well as 5-methyltetrahydrofolic acid (5-MTHF; a reduced folate form which can be prepared as either of four stereoisomers [6R-/6S- and L-Glu/D-Glu]) bind with high affinity to the FR (K_{D} in the nanomolar range), which internalizes via endocytosis (Kamen, B. A. et al., Adv Drug Deliv Rev 2004, 56, (8), 1085-97). Folic acid has, therefore, been used as a targeting agent to deliver attached diagnostic and therapeutic payloads for imaging and therapy of FR-expressing cancer (Low, P. S. et al., Acc Chem Res 2008, 41, (1)). Only a few of the developed folate radioconjugates were used in clinics, among those [¹¹¹In]In-DTPA-folate and [^{99m}Tc]Tc-EC20 (Etarfolatide™, Endocyte Inc.) for single photon emission computed tomography (SPECT) (Siegel, B. A. et al., J Nucl Med 2003, 44, (5), 700-7; Fisher, R. E. et al, J Nucl Med 2008, 49, (6), 899-906) and ¹⁸F-AzaFol (3′-aza-2′-[¹⁸F]fluorofolic acid) for positron emission tomography (PET) (Gnesin, S. et al., EJNMMI Res 2020, 10, (1), 32).

Based on preclinical studies in mice, the main concern with regard to a therapeutic application of folate radioconjugates refers to a potential risk of damage to the kidneys (radionephrotoxicity) as a result of the high renal accumulation of folate radioconjugates (Müller, C. et al., Nucl Med Biol 2011, 38, (5), 715-23). The incorporation of 4-(p-iodophenyl)butanoic acid (Dumelin, C. E. et al., Angew Chem Int Ed Engl 2008, 47, (17), 3196-201) as an albumin binder into the structure of folate radioconjugates ([¹⁷⁷Lu]Lu-cm09 (Müller, C. et al., J Nucl Med 2013, 54, (1), 124-31) and [¹⁷⁷Lu]Lu-cm10 (Müller et al, J Nucl Med 2014, 55, (10), 1658-64, herein referred to as [¹⁷⁷Lu]Lu-OxFol-1) had a positive effect on their pharmacokinetic properties. Due to the albumin-binding properties, the blood circulation of the folate radioconjugate was enhanced which resulted in increased tumor accumulation and reduced retention in the kidneys and, hence, significantly improved tumor-to-kidney ratios (Müller, C. et al., J Nucl Med 2013, 54, (1), 124-31; Siwowska, K. et al. Mol Pharm 2017, 14, (2), 523-532). The use of albumin-binding radioconjugates comprising 5-methyltetrahydrofolate (5-MTHF) as a targeting agent showed high tumor-to-kidney ratios and, as a consequence, a superior therapeutic effect as compared to the respective folic acid based compound [¹⁷⁷Lu]Lu-OxFol-1 (Guzik, P. et al., Eur J Nucl Med Mol Imaging 2021, 48, 972-983).

### SUMMARY OF THE DISCLOSURE

The present disclosure is in a first aspect directed to new folate-conjugates comprising a 5-methyltetrahydrofolate, a radiometal chelator optionally coordinating a radiometal M, and 5-(p-iodophenyl)pentanoate as an albumin binder.

In one specific embodiment, the new folate conjugates are compounds of formula I, or a stereoisomer (or a combination of stereoisomers) thereof, or a pharmaceutically acceptable salt thereof

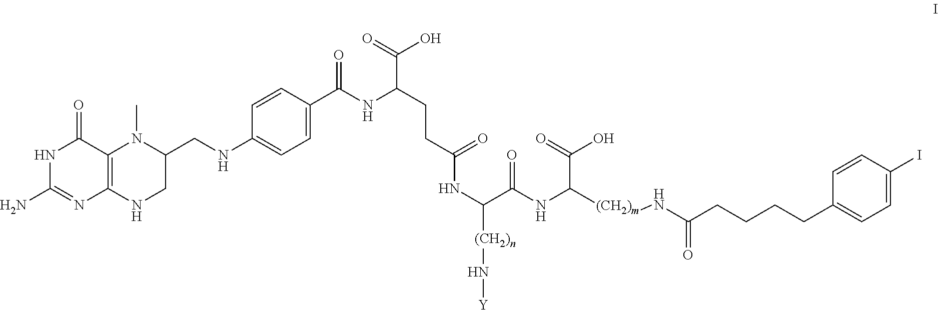

wherein Y is a radiometal chelator optionally coordinating a radiometal M, n is 1 to 8, and m is 1 to 8.

In some embodiments, the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, AAZTA, HP-DO3A, EDTA, TETA, EHPG, HBED, NOTA (and derivatives such as p-SCN-NOTA), DOTAGA, DOTMA, TETMA, PDTA, TTHA, LICAM, MECAM, AAZTA, preferably macrocyclic polyaminocarboxylates, such as NOTA, DOTA, DTPA, DO3A, HP-DO3A, EDTA, TETA, DOTMA, AAZTA. The radiometal chelator, e.g. the macrocyclic polyaminocarboxylate is covalently bound to a compound of the disclosure through amide coupling of one of its carboxylate groups.

The radiometal chelator may or may not be coordinating a radiometal M. In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu, ⁴³Sc, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, 90Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

In some embodiments, n is 2, 3, 4, 5 or 6, preferably 4. In some embodiments, m is 2, 3, 4, 5 or 6, preferably 4. In some embodiments, n is 4 and m is 4.

In a further aspect the present disclosure provides uses of a compound and/or pharmaceutical composition of the present disclosure in diagnostic imaging, radionuclide therapy or theragnostic applications. In some embodiments, the subject of the methods of the present disclosure is a mammal, such as an animal or a human. In some embodiments, the subject of the methods of the present disclosure is a human.

In a further aspect the present disclosure provides a single or multi-vial kit containing all of the components needed to prepare the compounds of this disclosure.

Other features and advantages of the disclosure will be apparent from the following detailed description thereof and from the claims.

It is to be understood that the description and the embodiments are intended to provide an overview or framework for understanding the nature and character of the disclosure without limiting its scope.

### BRIEF DESCRIPTION OF THE FIGURES

FIG. 1. Synthesis scheme of RedFol-24: a) Fmoc-Lys(Alloc)-OH, DIPEA in DMF; o/n; b) 50% Piperidine in DMF; 2×5 min; c) Dde-Lys(Fmoc)-OH, HBTU, DIPEA in DMF; 1 h; d) DOTA-tris(tBu) ester, HBTU, DIPEA in DMF; 3 h; e) Pd(PPh₃)₄, morpholine in DCM; 1 h; f) 5-(p-iodophenyl)pentanoic acid, HBTU, DIPEA in DMF; 1 h; g) 2% Hydrazine in DMF; 1.5 h; h) Fmoc-Glu-OtBu, HBTU, DIPEA in DMF; 1.5 h; i) 10-formyl-5-methyltetrahydropteroic acid, HBTU; DIPEA in DMF; 2 h; j) TFA, TIPS, H₂O (95:2.5:2.5); 2 h; k) aq. NaOH (1 M); 5 h.

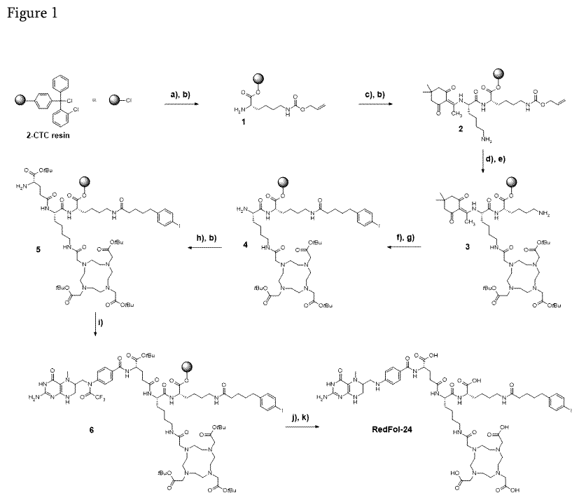

FIG. 2. Uptake and internalization of folate radioconjugates in KB tumor cells after an incubation period of 2 h and 4 h at 37° C. (A) Uptake of the folate radioconjugates in KB tumor cells. (B) Internalized fraction of folate radioconjugates in KB tumor cells.

FIG. 3. Decay-corrected uptake of the folate radioconjugates in KB tumor and various other tissues of tumor-bearing mice 1h, 4 h, 24h after injection.

FIG. 4. Tumor-to-blood ratios determined based on biodistribution data obtained at 1 h, 4 h and 24 h after injection of respective radioconjugates.

FIG. 5. Tumor-to-kidney ratios determined based on biodistribution data obtained at 1 h, 4 h and 24 h after injection of respective radioconjugates.

FIG. 6. Tumor-to-liver ratios determined based on biodistribution data obtained at 1 h, 4 h and 24 h after injection of respective radioconjugates.

FIG. 7. SPECT/CT images shown as maximum intensity projections (MIPs) of KB tumor-bearing mice 1 h, 4 h and 24 h after injection of the ¹⁷⁷Lu-folate radioconjugates (25 MBq; 0.5 nmol per mouse). (A) SPECT/CT scans of [¹⁷⁷Lu]Lu-OxFol radioconjugates; (B) SPECT/CT scans of 6R-5-MTHF-based radioconjugates; (C) SPECT/CT scans of 6S-5-MTHF-based radioconjugates; Tu=KB tumor; Ki=kidney; H=heart.

### DETAILED DESCRIPTION OF THE DISCLOSURE

The present disclosure is in a first aspect directed to new folate conjugates (hereinafter also called compounds or conjugates of the disclosure) comprising a 5-methyltetrahydrofolate (5-MTHF), a radiometal chelator optionally coordinating a radiometal M, and the albumin binder 5-(p-iodophenyl)pentanoate.

The term “radiometal chelator” (or (metal) chelator) may be any of the metal chelators known in the art for complexing a radiometal or radionuclide (and useful for the intended applications). The binding of a chelator to a radiometal may be determined by measuring the dissociation constant between chelator and radiometal. For the purposes of the present disclosure, the dissociation constant K_{D} between chelator and radiometal is from about 10⁻³ to about 10⁻¹⁵ M⁻¹. Preferably, the dissociation constant K_{D} between chelator and radiometal is from about 10⁻⁶ to about 10⁻¹⁵ M⁻¹. In some embodiments, a radiometal for use in the present disclosure is one, which can be detected externally in a non-invasive manner following administration in vivo. In some embodiments, the radiometal is particularly one which is suitable for imaging using SPECT or PET.

Examples of chelators are well known in the art, and include bidentate, tridentate, and tetradentate ligands in linear, tripodal and macrocyclic form. Typical examples include bipyridyl (bipy); terpyridyl (terpy); crown ethers; aza-crown ethers; succinic acid; citric acid; salicylic acids; histidines; imidazoles; ethyleneglycol-bis-(beta-aminoethyl ether) N,N′tetraacetic acid (EGTA); nitroloacetic acid; acetylacetonate (acac); sulfate; dithiocarbamates; carboxylates; alkyldiamines; ethylenediamine (en); diethylenetriamine (dien); nitrate; nitro; nitroso; (C₆H₅)₂PCH₂CH₂P(C₆H₅)₂ (diphos); glyme; diglyme; bis(acetylacetonate) ethylenediamine (acacen); ethylenediaminotetraacetic acid (EDTA), diethylenetriaminopentaacetic acid (DTPA); N-[2-[bis(carboxymethyl)amino]-3-(4-ethoxyphenyl)propyl]-N-[2-[bis(carboxymethyl)-amino]ethyl]-L-glycine (EOB-DTPA); N,N-bis[2-[bis(carboxymethyl)amino]-ethyl]-L-glutamic acid (DTPA-Glu); N,N-bis[2-[bis(carboxymethyl)amino]-ethyl]-L-lysine (DTPA-Lys); mono- or bis-amide derivatives of DTPA such as N,N-bis[2-[carboxymethyl[(methylcarbamoyl)methyl]amino]-ethyl]glycine (DTPA-BMA); N-[5-(Acetyl-hydroxy-amino)pentyl]-N-{5-[3-(5-aminopentyl-hydroxy-carbamoyl)propanoylamino]pentyl}-N-hydroxy-butandiamid (DFO); N1-(27-amino-11,22-dihydroxy-7,10,18,21-tetraoxo-6,11,17,22-tetraazaheptacosyl)-N1-hydroxy-N4-(5-(N-hydroxyacetamido)pentyl)succinamide (DFO*); 4-carboxy-5,8,11-tris(carboxymethyl)-1-phenyl-2-oxa-5,8,11-triazamidecan-13-oic acid (BOPTA); 2,2′-(6-(bis(carboxymethyl)amino)-6-(4-carboxybutyl)-1,4-diazepane-1,4-diyl) diacetic acid (AAZTA); 1,4,7,10-tetraazacyclododecan-1,4,7-triactetic acid (DO3A); 1,4,7,10-tetraazacyclododecan-1,4,7,10-tetraactetic acid (DOTA); 1,4,7,10-tetraazacyclododececane, 1-(glutaric acid)-4,7,10-triacetic acid (DOTAGA); 10-(2-hydroxypropyl)-1,4,7,10-tetraazacyclododecan-1,4,7-triacetic acid (HPDO3A); 2-methyl-1,4,7,10-tetraazacyclododecan-1,4,7,10-tetraacetic acid (MCTA); tetramethyl-1,4,7,10-tetraazacyclododecan-1,4,7,10-tetraacetic acid (DOTMA); 3,6,9,15-tetraazabicyclo[9.3.1]pentadeca-1(15); 11,13-triene-3,6,9-triacetic acid (PCTA); PCTA12; cyclo-PCTA12; N,N-Bis(2-aminoethyl)-1,2-ethanediamine (TETA); 1,4,7,10-tetraazacyclotridecane-N,N′,N″,N′″-tetraacetic acid (TRITA); 1,12-dicarbonyl, 15-(4-isothiocyanatobenzyl) 1,4,7,10,13-pentaazacyclohexadecane-N,N′,N″-triacetic acid (HETA); 1,4,7,10-tetraazacyclododecane-N,N′,N″,N′″-tetraacetic acid mono-(N-hydroxysuccinimidyl) ester (DOTA-NHS); N,N′-Bis(2-aminoethyl)-1,2-ethanediamine-N-hydroxy-succinimide ester (TETA-NHS); [(2S,5S,8S,11S)-4,7,10-tris-carboxymethyl-2,5,8,11-tetramethyl-1,4,7,10-tetraazacyclododecan-1-yl]acetic acid (M4DOTA); [(2,5,8S,11S)-4,7-bis-carboxymethyl-2,5,8,11-tetramethyl-1; 4,7,10-tetraazacyclo-dodecan-1-yl]acetic acid; (M4DO3A); (R)-2-[(2S,5S,8S,11S)-4,7,10-tris-((R)-1-carboxyethyl)-2,5,8,11-tetramethyl-1,4,7,10-tetraazacyclododecan-1-yl]propionic acid (M4DOTMA); 10-phosphonomethyl-1,4,7,10-tetraazacyclododecane-1,4,7-triacetic acid (MPDO3A); hydroxybenzyl-ethylenediamine-diacetic acid (HBED) and N,N′-ethylenebis-[2-(o-hydroxyphenolic)glycine](EHPG).

Suitable metal chelators for use in the compounds of the present disclosure include bidentate, tridentate, and tetradentate, ligands in linear, tripodal and macrocyclic form, as identified hereinabove. In some embodiments, the metal chelators used for the present disclosure include linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, MECAM. In some embodiments, the metal chelators used for the present disclosure include macrocyclic polyaminocarboxylates, such as NOTA, DOTA, DTPA, DO3A, HP-DO3A, EDTA, TETA, DOTMA, AAZTA. The radiometal chelator as defined herein is linked to a compound of the disclosure through one of its carboxylate groups, for example through coupling with an amino group to give an amide linkage.

The radiometal chelator may or may not be coordinating a radiometal M. As used herein, the term “radiometal” (also referred to as radionuclide) refers to an atom capable of undergoing radioactive decay and may be used as diagnostic imaging agents or therapeutic agents as described below. In some embodiments, radiometal for nuclear imaging or radionuclide therapy include ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu, ⁴³Sc, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

The choice of metal will be determined based on the intended therapeutic or diagnostic use. A skilled person will know which radiometal to choose for the intended application.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

The term “compounds of the disclosure” encompasses compounds of formula (I) to (III) and any stereoisomers thereof (such as explicitly the (6R)- or (6S)-isomers) and any pharmaceutically acceptable salt thereof.

The disclosure also encompasses compounds of the disclosure, in which one or more atoms are replaced by a specific isotope of the corresponding atom, e.g. in which one or more or all hydrogen atom are replaced by deuterium atoms D to form compounds of the disclosure that are enriched in deuterium.

In some embodiments, a compound of the disclosure is a compound of formula I or pharmaceutically acceptable salt or a stereoisomer thereof

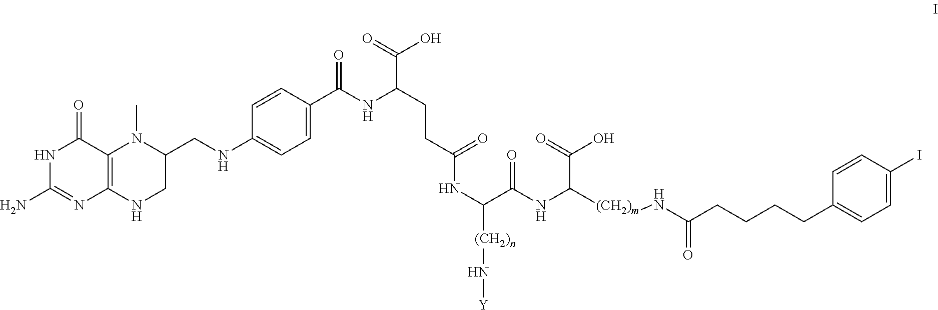

wherein
Y is a radiometal chelator optionally coordinating a radiometal M,
n is 1 to 8, and m is 1 to 8.

In some embodiments, the radiometal chelator is as defined hereinabove. In some embodiments, the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, MECAM.

In some embodiments, the metal chelators used for the present disclosure include macrocyclic polyaminocarboxylates, such as NOTA, DOTA, DTPA, DO3A, HP-DO3A, AAZTA, EDTA, TETA, DOTMA.

In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu ⁴³SC, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, 67Cu, ⁹⁰Y, ¹⁶¹Tb, ¹⁴⁹Tb, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

In some embodiments, n is 2, 3, 4, 5 or 6, preferably 4. In some embodiments, m is 2, 3, 4, 5 or 6, preferably 4. In some embodiments, wherein n is 4 and m is 4.

In some embodiments, a compound of formula I has the formula Ia or Ib

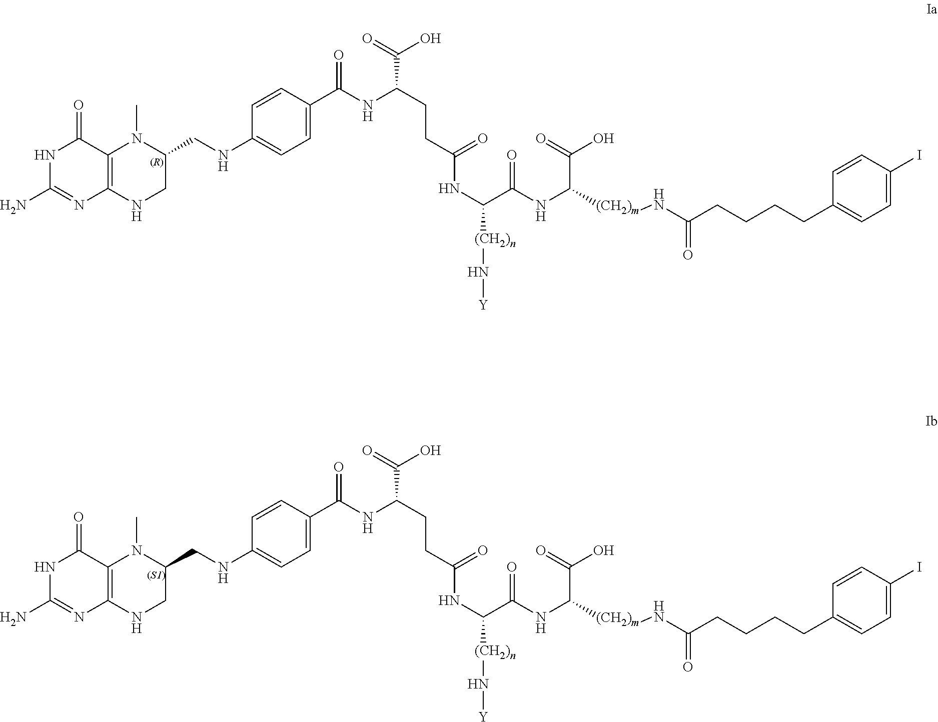

wherein
Y is a radiometal chelator optionally coordinating a radiometal M,
n is 1 to 8, and m is 1 to 8.

In some embodiments, the radiometal chelator is as defined hereinabove. In some embodiments, the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, MECAM.

In some embodiments, the metal chelators used for the present disclosure include macrocyclic polyaminocarboxylates, such as NOTA, DOTA, DTPA, DO3A, HP-DO3A, AAZTA, EDTA, TETA, DOTMA.

In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu, ⁴³Sc, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

In some embodiments, n is 2, 3, 4, 5 or 6, preferably 4. In some embodiments, m is 2, 3, 4, 5 or 6, preferably 4. In some embodiments, wherein n is 4 and m is 4.

In some embodiments, a compound of formula I has the formula II

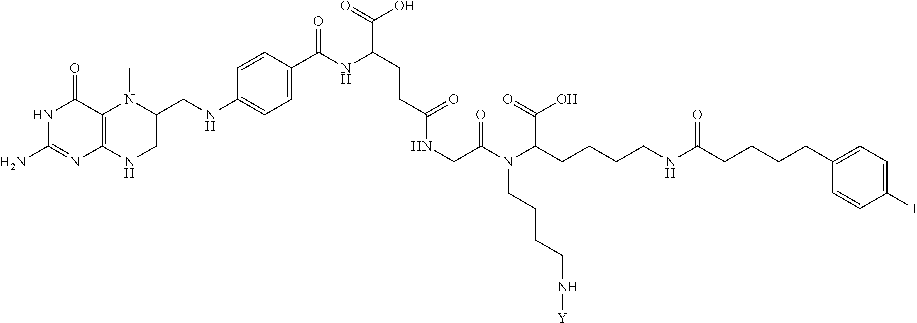

wherein Y is a radiometal chelator optionally coordinating a radiometal M.

In some embodiments, the radiometal chelator is as defined hereinabove. In some embodiments, the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, MECAM.

In some embodiments, the metal chelators used for the present disclosure include macrocyclic polyaminocarboxylates, such as NOTA, DOTA, DTPA, DO3A, HP-DO3A, AAZTA, EDTA, TETA, DOTMA.

In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Ph, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac ¹⁹⁸Au, ¹⁹⁹Au, and ²²⁷Th.

In some embodiments, the optionally coordinated radiometal M is selected from ⁶⁷Ga, ⁶⁸Ga, 64Cu, 67Cu, ⁴³SC, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³SC, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

In some embodiments, a compound of formula I has the formula IIa or IIb

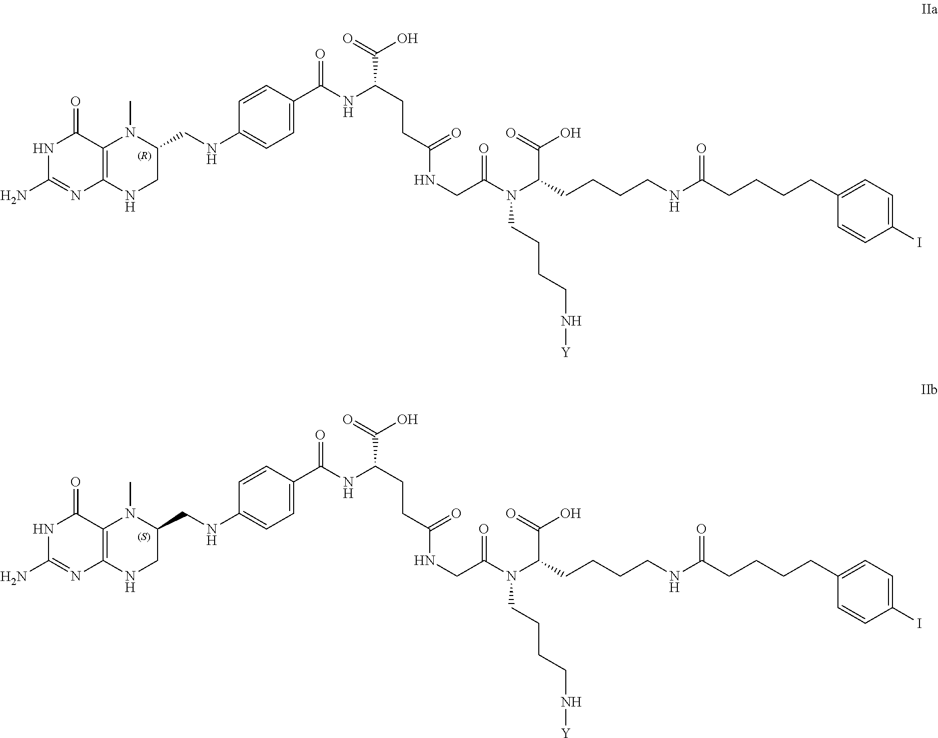

wherein Y is a radiometal chelator optionally coordinating a radiometal M.

In some embodiments, the radiometal chelator is as defined hereinabove. In some embodiments, the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, MECAM.

In some embodiments, the metal chelators used for the present disclosure include macrocyclic polyaminocarboxylates, such as NOTA, DOTA, DTPA, DO3A, HP-DO3A, AAZTA, EDTA, TETA, DOTMA.

In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu ⁴³SC, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

In some embodiments, a compound of formula I has the formula III and is optionally coordinating a radiometal M

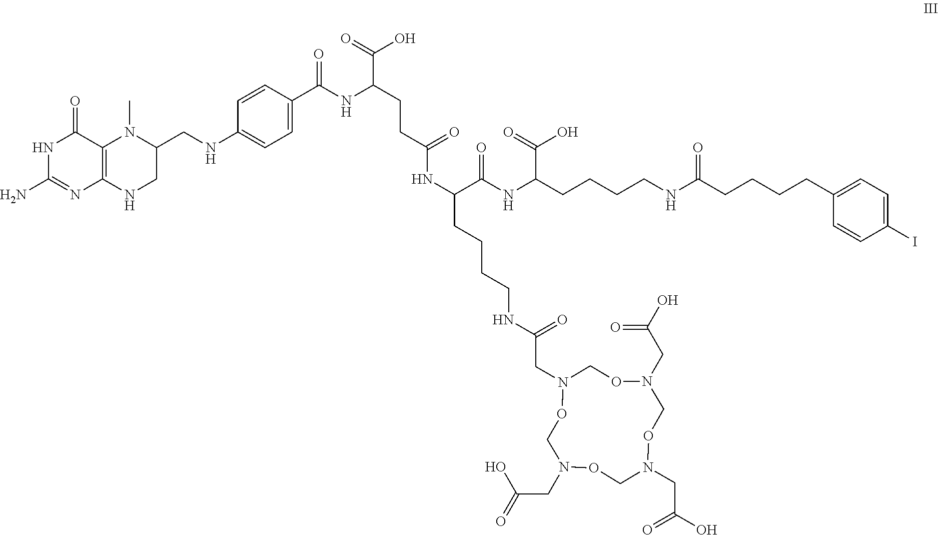 [SMILES: CN1c2c(nc(N)[nH]c2=O)NCC1CNc1ccc(C(=O)NC(CCC(=O)NC(CCCCNC(=O)CN2CON(CC(=O)O)CON(CC(=O)O)CON(CC(=O)O)CO2)C(=O)NC(CCCCNC(=O)CCCCc2ccc(I)cc2)C(=O)O)C(=O)O)cc1]

In some embodiments, the optionally coordinated radiometal M is selected from ⁵¹Cr, ⁶⁷Ga, ⁶⁸Ga, 4Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, 64Cu, 67Cu, ⁴³Sc, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, 64Cu, ⁴³Sc, ⁴⁴Sc, ^{99m}Tc, ¹⁵²Tb, ¹⁵⁵Tb, 67Cu, ⁹⁰Y, ¹⁶¹Tb, ¹⁴⁹Tb, ¹⁷⁷Lu, and ²²⁵Ac.

In some embodiments, the optionally coordinated radiometal M is for use in diagnostic imaging and is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M is for use in diagnostic imaging and is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M is for use in radionuclide therapy and is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb ¹⁷⁷Lu, ¹⁸⁶Re, ¹⁸⁸Re, ²²⁵Ac, and ²¹³Bi.

In some embodiments, the optionally coordinated radiometal M is for use in radionuclide therapy and is selected from ⁶⁷Cu, ⁹⁰Y ¹⁶¹Tb, ¹⁴⁹Tb, ¹⁷⁷Lu, and ²²⁵Ac.

In some embodiments, a compound of formula I has the formula IIIa or IIIb and is optionally coordinating a radiometal M

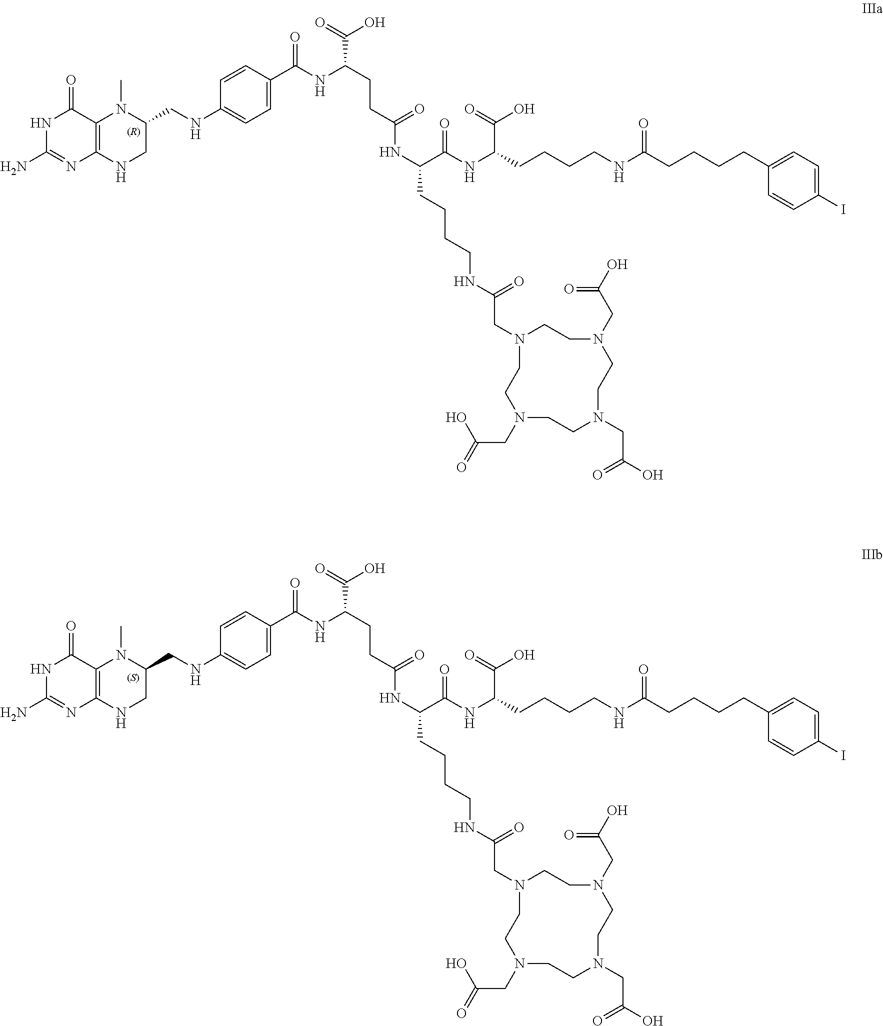

In some embodiments, the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu ⁴³SC, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

In some embodiments, the optionally coordinated radiometal M is for use in diagnostic imaging and is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M is for use in diagnostic imaging and is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M is for use in radionuclide therapy and is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M is for use in radionuclide therapy and is selected from ⁶⁷Cu, ⁹⁰Y, ¹⁶¹Tb, ¹⁴⁹Tb, ¹⁷⁷Lu, and ²²⁵Ac.

The compounds of the disclosure can be prepared by methods known in the field of synthetic chemistry and as described in the examples of the present disclosure.

In a further aspect the disclosure provides pharmaceutical compositions comprising a diagnostically effective amount or a therapeutically effective amount of at least one compound of the disclosure and a pharmaceutically acceptable carrier therefor. As used herein, a pharmaceutically acceptable carrier, which is present in an appropriate dosage, includes solvents, dispersion media, antibacterial and antifungal agents, isotonic agents, and the like, which are physiologically acceptable. The use of such media and agents are well-known in the art.

In a further aspect, the disclosure provides compounds and/or pharmaceutical compositions of the disclosure (i) for use as a diagnostic imaging agent, (ii) for use in radionuclide therapy or (iii) for use as a theragnostic agent, e.g. for use in targeting, visualization and treatment of a tumor, in particular in monitoring and assessing different stages, progression and migration of a tumor, to determine a suitable radionuclide therapy regimen (frequency, dosage etc.).

In some embodiments, the subject of the methods of the present disclosure is a mammal, such as an animal or a human. In some embodiments, the subject is a human.

In some embodiments, the compounds and/or pharmaceutical compositions of the disclosure may be used for diagnostic imaging, i.e. for use in diagnostic imaging of a cell or population of cells expressing a folate-receptor in vitro or in vivo, i.e. for convenient and effective administration to a subject in need for diagnostic imaging.

In some embodiments, the compounds and/or pharmaceutical compositions of the disclosure may be used for diagnostic imaging of a cell or population of cells expressing a folate-receptor comprising the steps of administering at least one compound and/or pharmaceutical compositions of the disclosure in diagnostically effective amounts and obtaining diagnostic image of the cell or population of cells expressing a folate-receptor.

In some embodiments, the compounds and/or pharmaceutical compositions of the disclosure may be used as a diagnostic imaging agent for therapeutic planning and/or monitoring the effectiveness of an ongoing therapeutic treatment.

In some embodiments, the present disclosure provides a method for diagnostic imaging of a cell or population of cells expressing a FR, said method comprising the steps of administering at least one compound or composition of the present disclosure in a diagnostically effective amount, and obtaining a diagnostic image of said cell or population of cells.

In some embodiments, the present disclosure provides a method for in vitro detection of a cell, e.g. a tumor cell, expressing the folate receptor in a tissue sample, e.g. a tissue biopsy taken from a subject, which includes contacting said tissue sample with a compound or composition of the present disclosure in diagnostically effective amounts and for sufficient time and conditions to allow binding to occur and detecting such binding by imaging techniques, such as PET imaging.

In some embodiments, the present disclosure provides a method for diagnostic imaging or monitoring (e.g. cancer therapy) a subject comprising the steps of (i) administering to the subject at least one compound and/or pharmaceutical composition of the present disclosure in a diagnostically effective amount, and (ii) performing diagnostic imaging using PET by detecting a signal from said at least one compound and/or pharmaceutical composition of the present disclosure (to follow the course of cancer therapy and/or determine a therapeutically effective amount of at least one further compound of composition of the disclosure to be administered for treatment).

In some embodiments, the compounds and/or pharmaceutical compositions of the present disclosure may be used for radionuclide therapy, i.e. for convenient and effective administration to a subject in need for radionuclide therapy.

In some embodiments, the compounds and/or pharmaceutical compositions of the present disclosure may be used for radionuclide therapy comprising the steps of administering to the subject in need thereof at least one and/or pharmaceutical composition of the present disclosure in therapeutically effective amounts, localizing the at least one compound and/or pharmaceutical composition in a tissue to be treated, and subjecting the tissue to radiation to achieve the desired therapeutic effect.

In some embodiments, the present disclosure provides a method for radionuclide therapy comprising the steps of administering to a subject in need thereof at least one compound or pharmaceutical composition of the present disclosure in therapeutically effective amounts, and after localization of said at least one compound or pharmaceutical composition in the desired tissues, subjecting the tissues to radiation to achieve the desired therapeutic effect.

In some embodiments, the compounds and/or pharmaceutical compositions of the present disclosure may be used as theragnostic agents for theragnostic applications.

The term “theragnostic” is derived from therapy and diagnostics and refers with regard to applications or agents, to the strategy of utilising the same radioactively labelled drug eventually containing a different radionuclide, for diagnostics and for therapy. This allows to take images of a disease with a compound of the disclosure coordinated to a radionuclide effective for diagnostic and treatment planning purposes. It is then possible to treat the disease by changing to a radionuclide effective for tumor treatment. This is the so called ‘treat what you see’ principle. More specifically, the same compound of the disclosure may be used as a theragnostic agent first as a diagnostic imaging agent (a “diagnostic compound of the disclosure”) in a diagnostically effective amount with a radionuclide effective for tumor localization, assessment, monitoring or therapy planning and second as a therapeutic agent (a “therapeutic compound of the disclosure”) in a therapeutically effective amount with a radionuclide effective for tumor treatment. Combining both functions allows optimization of selectivity (i.e. biodistribution) and efficacy (i.e. effective dosage), e.g. by first localizing and monitoring a tumor or cancerous tissue using a diagnostic compound of the disclosure, and subsequently tailoring a suitable administration regimen (i.e. frequency, dosage, etc.) of a therapeutic compound of the present disclosure according to the needs of an individual subject.

Thus in some embodiments, the compounds of the present disclosure may be used in the therapeutic planning and/or treatment and/or monitoring of a tumor (or cancerous tissue) by administering to a subject in need thereof (i) at least one compound or composition of the disclosure in a diagnostically effective amount (i.e. an amount effective to obtain a diagnostic image and/or for treatment planning, i.e. establishing a treatment regimen), and (ii) at least one further compound or composition of the disclosure in a therapeutically effective amount for tumor treatment (by subjecting the tumor (or cancerous tissue) to radiation) to achieve the desired therapeutic effect.

The diagnostically effective amount is an amount effective for diagnostic imaging, i.e. an amount effective to obtain a diagnostic image for treatment planning, i.e. an amount effective to obtain a diagnostic image based on which a treatment regimen can be designed (i.e. the therapeutically effective amount of the at least one further compound or composition of the disclosure to be administered can be determined or calculated) or an ongoing treatment regimen can be modified.

In some embodiments, the at least one compound or composition of the present disclosure in a diagnostically effective amount is a diagnostic compound coordinating a radiometal M for use in diagnostic imaging as defined herein.

In some embodiments, the at least one further compound or composition of the present disclosure in a therapeutically effective amount is a therapeutic compound coordinating a radiometal M for use in radionuclide therapy as defined herein.

A “diagnostically effective amount” of a compound or composition of the present disclosure to be administered is an amount sufficient to produce a diagnostic image of a tumor, a cancerous tissue, an organ or other site of the subject and/or an amount sufficient to determine the therapeutically effective amount for a treatment. A diagnostically effective amount of a compound or composition of the present disclosure is administered to monitor tumor growth or size before, during and after radionuclide therapy, and allows planning, tailoring and adjusting the therapy during the course of a treatment. In theragnostic applications, obtained results of the administration of a diagnostically effective amount of a compound or composition of the present disclosure are used to calculate the therapeutically effective amount.

A “therapeutically effective amount” of a compound or composition of the present disclosure to be administered is an amount sufficient to produce a desired radiotherapeutic effect. More specifically, a therapeutically effective amount is an amount of at least one of the compounds of the present disclosure sufficient to substantially improve, i.e. ameliorate, decrease or suppress, at least one symptom associated with the disease or condition, and/or to delay, hinder, or prevent the onset of the disease or condition.

In some embodiments, a diagnostic compound of the present disclosure is a compound of the present disclosure wherein the radiometal M is a diagnostic radionuclide permitting diagnosis of a tumor. In some embodiments a diagnostic radionuclide is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb. In some embodiments, a diagnostic radionuclide is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, a therapeutic compound of the present disclosure is a compound of the present disclosure wherein the radiometal M is a therapeutic radionuclide permitting treatment of a tumor. In some embodiments a therapeutic radionuclide is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi. In some embodiments, a therapeutic radionuclide is selected from ⁶⁷Cu, ⁹⁰Y, ¹⁶¹Tb, ¹⁴⁹Tb, ¹⁷⁷Lu, and ²²⁵Ac.

It is understood that a diagnostic compound of the present disclosure and a therapeutic compound of the present disclosure are administered to a subject sequentially.

In some embodiments, the present disclosure provides a method for therapeutic planning of a treatment in a subject comprising the steps of (i) administering to a subject in need thereof at least one compound or composition of the present disclosure in diagnostically effective amounts to obtain a diagnostic image, and (ii) determining the therapeutically effective amount of at least one further compound or composition of the present disclosure to be administered for treatment.

In some embodiments, the present disclosure provides a method for a theragnostic application in a subject comprising the steps of (i) administering to a subject in need thereof at least one compound or composition of the present disclosure in diagnostically effective amounts to obtain a diagnostic image, and (ii) administering at least one further compound or composition of the present disclosure in therapeutically effective amounts for treatment (i.e. by subjecting the tissue to radiation to achieve the desired therapeutic effect).

In some embodiments, the diagnostic image obtained of the at least one compound or composition in the tissue to be treated is used to determine (or calculate) the therapeutically effective amount of the compound or composition of the present used in for treatment.

In some embodiments, the present disclosure provides a method for a theragnostic application, i.e. encompassing both diagnosis and radionuclide therapy, comprising the steps of (i) administering to a subject in need thereof at least one compound or composition of the present disclosure in a diagnostically effective amount, (ii) obtaining a diagnostic image of the at least one compound or composition in the tissue to be treated, (iii) administering at least one further compound or composition of the present disclosure in a therapeutically effective amount and (iv) subjecting the tissue to radiation, to achieve the desired therapeutic effect.

In some embodiments, the at least one compound or composition of the present disclosure in a diagnostically effective amount is a diagnostic compound coordinating a radiometal M for use in diagnostic imaging as defined herein.

In some embodiments, the at least one further compound or composition of the present disclosure in a diagnostically effective amount is a therapeutic compound coordinating a radiometal M for use in radionuclide therapy as defined herein.

In some embodiments, the diagnostic image of the at least one compound or composition in the tissue to be treated is used to determine (e.g. calculate) the therapeutically effective amount of the at least one further compound or composition administered to obtain a therapeutic effect.

An image of a cell or tissue expressing the FR, i.e. a tumor cell or tissue, labeled with one or more of the compounds or compositions of the present disclosure can be detected using a radiation detector, e.g. a γ-radiation detector. One such procedure utilizes scintigraphy. Tomographic imaging procedures, such as SPECT, can also be used to improve visualization. Selection and use of such radiation detectors is within the skill of one of ordinary skill in the art.

It is understood that the specific activity of the radioimaging metal ion of choice, e.g. include ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th, will be taken into consideration in determining a dosage for diagnostic imaging or radionuclide therapy.

In some embodiments, the unit dose to be administered has a radioactivity of about 0.1 MBq to about 104 MBq. In some embodiments, the unit dose to be administered for diagnostic imaging has a radioactivity of about 1 MBq to about 1′000 MBq, such as about 100 MBq to 600 MBq. In some embodiments, the unit dose to be administered for radionuclide therapy has a radioactivity of about 1′000 MBq to about 104 MBq, such as about 5′000 MBq to 8′000 MBq. For a solution to be injected a preferred unit dosage is from about 0.01 mL to about 10 mL. After e.g. intravenous administration, imaging of the organ or tumor in vivo can take place, if desired, from within minutes to hours or even longer, after the radiolabeled reagent has been administered to a subject.

The compounds and/or compositions of the present disclosure may be administered by an appropriate route such as parentally (for example, intravenously), intramuscularly or intraperitoneally or by any other suitable method. For example, the compounds and/or compositions of this disclosure may be administered to a subject by bolus or slow infusion intravenous injection. The suitable forms for injection include sterile aqueous solutions or dispersions and sterile powders of the above mentioned compounds and/or compositions of the present disclosure.

The compounds or pharmaceutical compositions are generally sterile. Sterilization can be accomplished by any art recognized technique, including but not limited to, sterile filtration, addition of antibacterial of antifungal agents, for example, paraben, chlorobutanol, phenol, sorbic acid, thimerosal, and the like.

Samples can be collected by procedures known to the skilled person, e.g., by collecting a tissue biopsy or a body fluid, by aspirating for tracheal or pulmonary samples and the like. Tissue samples to be tested include any tissue suspected to contain a cell expressing a FR, such as tumor cells, epithelial cells, kidneys, gastrointestinal or the hepatobiliary system, and others. Samples can be sectioned, e.g., with a microtome, to facilitate microscopic examination and observation of bound complex. Samples can also be fixed with an appropriate fixative either before or after incubation with one of the compounds or compositions of the present disclosure to improve the histological quality of sample tissues.

Time and conditions sufficient for binding of a complex of the present disclosure to a FR on the cell include standard tissue culture conditions, i.e. samples can be cultured in vitro and incubated with one of the compounds or compositions of the present disclosure in physiological media. Such conditions are well known to the skilled person. Alternatively, samples can be fixed and then incubated with a complex or composition of the present disclosure in an isotonic or physiological buffer.

A typical amount of said complex of the present disclosure for in vitro detection of a tumor cell can range from about 1 ng/L to about 1′000 μg/L. In some embodiments, the amount is about 1 μg/l to about 100 μg/L.

In some embodiments, the compounds of the disclosure for use in in vitro diagnosis of a tumor cell and for use in in vivo applications, are compounds of the disclosure wherein the radiometal chelator is coordinating a radiometal M moiety selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

In some embodiments, the optionally coordinated radiometal M is selected from ⁶⁷Ga, ⁶⁸Ga, ⁶⁴Cu, ⁶⁷Cu ⁴³Sc, ⁴⁴Sc, ^{99m}Tc, ¹⁴⁹Tb, ¹⁵²Tb, ¹⁵⁵Tb, ¹⁶¹Tb, ⁹⁰Y, ¹⁷⁷Lu, and ²²⁵Ac.

In some embodiments, the optionally coordinated radiometal M is selected from ⁶⁷Ga, ⁶⁸Ga, ⁶⁴Cu, ⁴³Sc, ⁴⁴Sc, ^{99m}Tc, ¹⁵²Tb, ¹⁵⁵Tb, ⁶⁷Cu, ⁹⁰Y, ¹⁶¹Tb, ¹⁴⁹Tb, ¹⁷⁷Lu, and ²²⁵Ac.

In some embodiments, the optionally coordinated radiometal M for use in diagnostic imaging is selected from ⁶¹Cu, ⁶²Cu, ⁶⁴Cu, ⁶⁷Ga, ⁶⁸Ga, ⁴³Sc, ⁴⁴Sc, ⁵²Mn, ⁸⁹Zr, ^{99m}Tc, ¹¹¹In, ¹⁵²Tb, and ¹⁵⁵Tb.

In some embodiments, the optionally coordinated radiometal M for use in diagnostic imaging is selected from ⁶⁷Ga, ⁶⁸Ga, ⁶⁴Cu, ⁴³Sc, ⁴⁴Sc, ^{99m}Tc, ¹⁵²Tb, and ¹⁵⁵Tb.

In some embodiments, the optionally coordinated radiometal M for use in radionuclide therapy is selected from ⁶⁷Cu, ⁹⁰Y, ¹⁰⁵Rh, ^{117m}Sn, ¹⁴⁹Pm, ¹⁵³Sm, ¹⁶¹Tb, ¹⁴⁹Tb, ¹⁶⁶Dy, ¹⁶⁶Ho, ¹⁷⁵Yb, ¹⁷⁷Lu, ¹⁸⁶Re, ¹⁸⁸Re, ²²⁵Ac, and ²¹³Bi.

In some embodiments, the optionally coordinated radiometal M for use in radionuclide therapy is selected from ⁶⁷Cu, ⁹⁰Y, ¹⁶¹Tb, ¹⁴⁹Tb, ¹⁷⁷Lu, and ²²⁵Ac.

For detection of cellular binding of one of the compounds of the disclosure, samples can be incubated in the presence of a compound, then washed and counted in a standard scintillation counter. Alternative methods apply and are known to the skilled person.

It is understood that the above methods of the disclosure may be performed in combination with any other methods of cancer diagnosis or therapy including methods using other already developed diagnostic and/or therapeutic agents and utilizing x-ray computed tomography (CT), magnetic resonance imaging (MRI), functional magnetic resonance imaging (fMRI), SPECT, optical imaging, and ultrasound.

For diagnostic imaging or radionuclide therapy or theragnostic applications it may be convenient to prepare the compounds of the present disclosure at, or near, the site where they are to be used. Thus the present disclosure provides in a further aspect a single or multi-vial kit containing all of the components needed to prepare compounds or compositions of this disclosure, other than the radiometal ion itself. Thus a preferred single-vial kit of the present disclosure comprises a compound of the present disclosure, without the radiometal chelator being coordinated, and a source of a pharmaceutically acceptable reducing agent such as a stannous salt. In addition, the kit comprises optionally further additives, for example the kit is buffered with a pharmaceutically acceptable acid or base to adjust the pH to a desired value for complex formation. Such a single vial kit may optionally contain exchange ligands such as glucoheptonate, gluconate, mannitol, maleate, citric or tartaric acid and may also contain reaction modifiers, such as diethylenetriaminepentaacetic acid or ethylenediamine tetraacetic acid. Additional additives, such as solubilizers (for example a cyclodextrin), antioxidants (for example ascorbic acid) and/or fillers (for example, NaCl) may be employed to improve the radiochemical purity and stability of the final product, or to aid in the production of the kit. The radiometal will typically be added separately in the form of a solution.

Likewise, a preferred multi-vial kit of the present disclosure comprises, in one vial, the components, other than the radiometal itself, that is, an exchange ligand and a pharmaceutically acceptable reducing agent such as a stannous salt. A compound of the present disclosure, wherein the radiometal chelator, is contained in a second vial, as well as optional additives such as buffers appropriate to adjust the pH to its optimal value.

Optionally, the radiometal will be provided in form of a solution to be added.

All components of a kit may be in liquid, frozen or dry form. In some embodiment, the kit components are provided in lyophilized form.

All of the compounds, compositions and/or methods disclosed and claimed herein can be made and executed without undue experimentation in light of the present disclosure. It will be apparent to those of skill in the art that variations may be applied to the present disclosure without departing from the scope of the disclosure. The Examples provided herein are intended to be illustrative and are not exhaustive; thus, the illustrated Examples should not be viewed as limiting the disclosure in any way.

### EXAMPLES

Methods: General: All commercially available solvents and chemicals were purchased from abcr, Bachem, Fluka, CheMatech, Iris Biotech, Merck KGaA or Sigma Aldrich and were used without further purification. The folate and pteroate precursors were obtained from Merck & Cie, Schaffhausen, Switzerland.

Cleavage from the resin and deprotection. Cleavage from the resin was performed using 2% trifluoroacetic acid (TFA) in dichloromethane (DCM) (v/v) followed by removal of the tri-tert-butyl and formyl protecting group in phosphoric acid (60%, aq., 24 h). Sodium hydroxide (NaOH) solution (32%, w/v, aq.) was added until pH ca. 4. Subsequent addition of HCl solution (1 M, aq.) to obtain pH 4 resulted in the precipitation of the final product. After centrifugation of the mixture at 14000 rpm for 5 min, the supernatant was discarded and the crude folate conjugate was re-dissolved and purified using RP-HPLC.

Purification of the folate conjugates. Purification of the final products was carried out using a Merck-Hitachi LaChrom HPLC system, equipped with a D-7000 interface, a L-7200 autosampler, a L-7400 UV detector, a L-7100 pump and a semi-preparative reversed-phase C18 column (5 μm, 10×150 mm, Sunfire™, Waters, Milford, US-MA). The products were eluted using variable gradient conditions in Milli-Q water/acetonitrile (MeCN)/TFA or Milli-Q water/MeCN/NH₄HCO₃ systems at a flow rate of 4 mL/min. The fractions containing the pure product were collected in a round-bottom flask, frozen in liquid nitrogen and lyophilized for 24 h.

Characterization of the folate conjugates. The final products were characterized using high resolution MALDI-TOF-MS (Bruker UltraFlex II; Billarica, US-MA).

Radiolabeling: The folate conjugates were dissolved in Milli-Q water containing 4-6% sodium L-ascorbate (0.5 M) to obtain a final folate concentration of 1 mM, which was frozen at −20° C. as a stock solution. The conjugates were labeled with lutetium-177 (no-carrier-added, in 0.04 M HCl; ITM Medical Isotopes GmbH, Munich, Germany) up to a molar activity of 50 MBq/nmol. Radiolabeling was performed after defrosting the stock solution in a mixture of sodium acetate (0.5 M) and HCl (0.05 M) and ascorbic acid (0.5M) at pH ca. 4.5. The reaction mixture was incubated at 95° C. for 10-15 min.

Quality control: Aliquots of the radiolabeled conjugates were diluted in Milli-Q water containing penta-sodium diethylenetriamine pentaacetic acid (Na₅-DTPA; 50 μM) and assessed using a Merck Hitachi LaChrom HPLC system consisting of a L-7100 pump, a D-7000 interface, a L-7200 autosampler, a radioactivity detector (LB 506 B, Berthold Technologies GmbH) and a reversed-phase C18 column (5 μm, 4.6×150 mm, Xterra™ Waters, USA). A linear gradient of Milli-Q water containing 0.1% TFA (95-20% over 15 min) in MeCN (5-80% over 15 min) was used at a flow rate of 1.0 mL/min.

Radiolytic stability: After performing quality control of the radioconjugates (50 MBq/nmol) using HPLC (t₀=0 h), the radioconjugates were diluted in PBS pH 7.4 to obtain an activity concentration of 100 MBq/500 μL. The radioconjugate dilutions were incubated at room temperature. The integrity of the radioconjugates was assessed after a 4 h and 24 h, respectively, using HPLC. The HPLC chromatograms were analyzed by determination of the peak area of the radiolabeled product, the released lutetium-177 as well as the degradation products of unknown structure. The quantity of the intact product was expressed as the percentage of the sum of integrated peak areas of the entire chromatogram and set into relation to the original value determined at to (radiochemical purity >95% set as 100%).

### Example 1: Solid-Phase Synthesis of 6R-RedFol-24 and 6S-RedFol-24

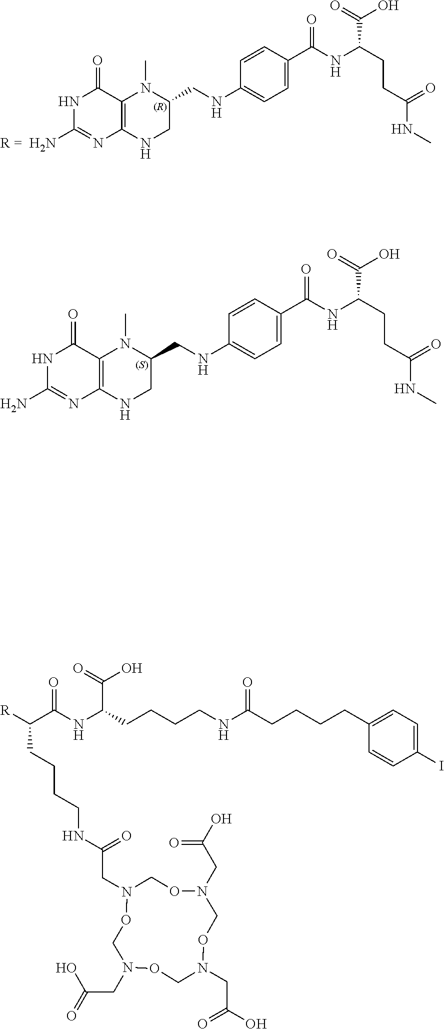

2-Chlorotrityl chloride (2-CTC) resin (0.1 mmol) was weighed into a filter-containing 5 mL-syringe and swelled in anhydrous DCM for 45 min. Na-fluorenylmethyloxycarbonyl-NE-(4-allyloxycarbonyl)-L-lysine (Fmoc-Lys(Alloc)-OH (0.12 mmol, 1.2 equiv)) was dissolved in dry DCM in the presence of diisopropylethylamine (DIPEA, 0.8 mmol, 8.0 equiv), added to the resin, and stirred overnight (o/n). Residual reactants were removed after each reaction step by washing the resin three times with dimethylformamide (DMF) or DCM depending on the utilized solvent. Potential unreacted carbocations of the 2-CTC resin were capped with a solution of DCM, methanol, and DIPEA (17:2:1, v/v). After conditioning in DMF, the Fmoc protecting group was removed by shaking in a mixture of DMF and piperidine in a ratio of 1:1 (v/V) two times for 5 min to yield compound 1. Then, Na-1-(4,4-dimethyl-2,6-dioxocyclohex-1-ylidene)ethyl-Nε-Fmoc-L-lysine (Dde-Lys(Fmoc)-OH, 0.4 mmol, 4.0 equiv) was activated for one minute with O-(benzotriazol-1-yl)-N,N,N′,N′tetramethyluronium-hexafluorophosphate (HBTU, 0.396 mmol, 3.96 equiv) in the presence of DIPEA (0.8 mmol, 8.0 equiv) in dry DMF, added to resin-immobilized compound 1, and reacted for 1 h. After removal of the Fmoc protecting group to obtain compound 2, activated tri-tert-butyl 1,4,7,10-tetraazacyclododecane-1,4,7,10-tetraacetate (DOTA-tris(tert-butyl) ester, 0.3 mmol, 3.0 equiv) was coupled to resin-immobilized compound 2 over the course of 3 h. Cleavage of the Alloc protecting group of the lysine residue was performed with tetrakis(triphenylphosphine)palladium(0) (Pd(PPh₃)₄, 0.045 mmol, 0.45 equiv) in the presence of morpholine (1.0 mmol, 10 equiv) in dry DCM within 1 h in the dark. To remove residuals of palladium, the resin-immobilized compound was washed with 1% DIPEA in DMF (v/v) and a solution of sodium diethyldithiocarbamate (35 mg/mL) in DMF to yield resin-immobilized compound 3.

Activated 5-(p-iodophenyl)pentanoic acid (0.4 mmol, 4.0 equiv) was added to compound 3 and agitated for 1 h before removal of the Dde protecting group by agitating in a hydrazine solution (2% in DMF, v/v) for 1.5 h to give compound 4. Compound 5 was obtained by coupling activated Fmoc-L-glutamic acid 5-tert-butyl ester (Fmoc-Glu-O(tert-butyl), 0.4 mmol, 4.0 equiv) for 1.5 h and removal of the Fmoc protecting group. 10-formyl-5-methyl-(6S)-tetrahydropteroic acid (6S-5-MTHP) was activated, added to resin-immobilized compound 5, and agitated for 2 h. The resultant resin-immobilized compound 6 was washed with DMF, DCM, and diethyl ether (Et₂O) and dried under reduced pressure. Cleavage from the resin and removal of the protecting groups were carried out as described below. The precipitate was dissolved in a mixture of MeCN and aqueous ammonium hydrogen solution (aq. NH₄HCO₃, 50 mM; 1:1, v/v) for purification using reversed-phase high performance liquid chromatography (RP-HPLC).

The synthesis of the reduced folate conjugates 6S-RedFol-24 was performed according to the procedure described for 6R-RedFol-24 except that instead of 10-formyl-5-methyl-(6S)-tetrahydropteroic acid (6S-5-MTHP) 10-formyl-5-methyl-(6R)-tetrahydropteroic acid (6R-5-MTHP) was conjugated to the resin-immobilized joint precursor under nitrogen atmosphere and exclusion of light.

### Example 2: Characterization of the Folate Conjugates of the Disclosure

TABLE 1
Chemical characterization of the folate conjugate (*: measured by
MALDI-MS and detected as [M + H]⁺; **: measured by
ESI-MS and detected as [M + 2H]2+; n.d. = not determined).

| Compound | m/Z_{calc} | m/Z_{found} | Yield [%] | Purity [%] |
|---|---|---|---|---|
| 6R-RedFol-24** | 694.78 | 694.27 | 4 | >95 |
| 6S-RedFol-24** | 694.78 | 694.78 | 2 | >93 |

### Example 3: Stability of the Folate Radioconjugates

TABLE 2
The product peak was expressed as
percentage of the sum of integrated peak areas
of the entire chromatogram (set as 100%) relative
to the value obtained immediately after
labelling (average of n = 2-3 individually performed experiments).

| | Intact radiolabeled | | |
|---|---|---|---|
| | product [%] | | |
| Radioconjugate | 4 h | 24 h | n |
| [¹⁷⁷Lu]Lu-6R-RedFol-24 | ≥99 | ≥99 | 2 |
| [¹⁷⁷Lu]Lu-6S-RedFol-24 | ≥99 | ≥99 | 2 |

### Example 4: PBS/n-Octanol Distribution Coefficient (Log D Values)

The n-octanol/PBS distribution coefficients (log D values) were determined for each folate radioconjugate of the disclosure in order to assess their hydrophilic/hydrophobic properties. The log D values of the folate radioconjugates of the disclosure (50 MBq/nmol) were determined as previously reported, after dilution in PBS pH 7.4 to obtain an activity concentration of 10 MBq/500 μL. A sample of each radiofolate (ca. 0.5 MBq, 25 μL, 0.01 nmol) was added to a mixture of 1475 μL PBS pH 7.4 and 1500 μL n-octanol. The vials were vortexed vigorously for 1 min followed by centrifugation for 6 min at 2500 rpm for phase separation. An aliquot was taken from each phase and measured in a gamma-counter (Perkin Elmer, Wallac Wizard 1480). The distribution coefficients were calculated as the logarithm of the ratio of counts per minute (cpm) measured in the n-octanol phase relative to the cpm measured in the PBS phase. The results were listed as mean±SD of the data obtained from 3 independent experiments, each performed with five replicates.

The log D values of the folate radioconjugate of the disclosure indicate their commonly hydrophilic character (Table 3). Yet the hydrophobicity of the radioconjugate of the disclosure was increased due to the additional methyl group in the 5-MTHF-based targeting molecule and the additional methylene group in the 5-(p-iodophenyl)pentanoate unit.

TABLE 3
LogD values of the folate
radioconjugates of the disclosure.

| Compound | m/Z_{calc} | m/Z_{found} |
|---|---|---|
| [¹⁷⁷Lu]Lu-6.R-RedFol-24 | −3.5 ± 0.2 | 3 |
| [¹⁷⁷Lu]Lu-6S-RedFol-24 | −3.1 ± 0.1 | 3 |

### Example 5: Albumin-Binding Properties (Filter Assay)

Albumin-binding properties were determined in mouse and human blood plasma. The albumin-binding properties of the folate radioconjugates of the disclosure in mouse blood plasma (Rockland Immunochemicals, Inc., USA) and human blood plasma (Stiftung Blutspende SRK Aargau-Solothurn, Switzerland) were determined using an ultrafiltration method (Deberle, L. M. et al., Molecules 2020, 25, (11)). The amount of serum albumin in mouse (MSA) and human (HSA) blood plasma was defined as 550 μM and 800 μM, respectively, based on measurements using a dry chemistry analyzer (DRI-CHEM 4000i, FUJIFILM, Japan). The folate radioconjugates of the disclosure (50 MBq/nmol, ca. 300 kBq, 0.006 nmol in 15 μL) were added to samples of mouse and human blood plasma (150 μL), followed by incubation of the samples at 37° C. for 30 min. The samples were loaded on Amicon centrifugal filters (cut-off of 10 kDa; Merck Millipore) followed by centrifugation (14,000 rcf, 30 min, 4° C.) to allow the separation of the plasma-bound from the plasma-unbound (free) fractions of each sample. The inserts of the filter devices were inverted and centrifuged at 200 rcf for 3 min to recover the protein-bound fraction. The activity of the protein-bound fraction as well as the activity in the filtrate and in the filter unit were measured separately in a gamma-counter (Perkin Elmer, Wallac Wizard 1480). The percentage of radioconjugate bound to mouse and human albumin, respectively, was set in relation to the total activity measured (Table 4).

TABLE 4
Albumin-bound fraction of the folate radioconjugates
of the disclosure in mouse and
human blood plasma. The data are presented as
the average of the albumin-bound fraction of 3 experiments.

| | Mouse blood | Human blood | |
|---|---|---|---|
| Radioconjugate | plasma [%] | plasma [%] | n |
| [¹⁷⁷Lu]Lu-6R-RedFol-24 | 73 | 94 | 3 |
| [¹⁷⁷Lu]Lu-6S-RedFol-24 | 74 | 86 | 3 |

### Example 6: Cell Uptake and Internalization

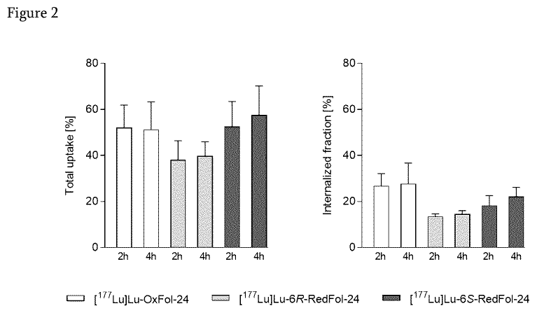

The experiments were performed as previously reported (Deberle, L. M. et al; Bioconj Chem 2021, 32, p. 1617). KB tumor cells (human cervical cancer cell line, ACC-136) were seeded in 12-well plates (0.5×10⁶ cells in 2 mL per well) using folate-free RPMI (FFRPMI) medium supplemented with 10% fetal calf serum, L-glutamine, antibiotics. The tumor cells were incubated overnight to allow adhesion and growth overnight at 37° C. and 5% CO₂. After removal of the supernatant, the KB cells were rinsed with PBS prior to the addition of FFRPMI medium without supplements (975 μL/well). The folate radioconjugates of the disclosure (50 MBq/nmol) were added to each well in a volume of 25 μL (0.75 μmol, 38 kBq). In some wells, KB tumor cells were co-incubated with excess folic acid (100 μM) to block the FRs on the cell surface. After incubation of the well plates for 2 h or 4 h at 37° C. and 5% CO₂, the KB tumor cells were rinsed three times with ice-cold PBS to determine total uptake of the folate radioconjugates of the disclosure. In order to assess the internalized fraction, a stripping buffer (solution of 0.1 M acetic acid and 0.15 M NaCl, aq., pH 3) was applied to release FR-bound folate radioconjugates of the disclosure from the cell surface. Cell samples were lysed by addition of NaOH solution (1 M, aq., 1 mL) to each well. The cell lysates were counted for activity in a gamma-counter (Perkin Elmer, Wallac Wizard 1480). After homogenization of the cell suspensions by vortexing, the protein concentrations of each sample were determined using a Micro BCA Protein Assay kit (Pierce, Thermo Scientific) in order to standardize the measured activity to the average content of protein in a single well. The uptake and internalized fraction was expressed as the percentage of total added activity and presented as the average ±SD of n=2-6 independent experiments (FIG. 2A/B).

### Example 7: FR-Binding Affinity (K_{D} Values)

The determination of the K_{D} values was performed as previously reported (Deberle, L. M. et al; Bioconj Chem 2021, 32, p. 1617). FR-positive IGROV-1 cells (human ovarian carcinoma cell line, kindly provided by Dr. Gerrit Janssen, Free University Medical Center Amsterdam, The Netherlands) were seeded in 48-well plates (2.5×10⁵ cells/well) in 500 μL FFRPMI medium with supplements. The cells were incubated at 37° C. and 5% CO₂ to allow cell adhesion overnight. The experiment was performed on ice using ice-cold medium and buffer solutions. After removal of the supernatant, the cells were rinsed once with PBS prior to the addition of FFRPMI medium without supplements (450 μL/well). The respective folate radioconjugate of the disclosure (20 MBq/nmol) was added to each well at variable concentrations (in the range of 0.1 to 500 nM, 50 μL per well). In order to determine the unspecific binding of the folate radioconjugates of the disclosure, half of the cell samples were co-incubated with excess folic acid (100 μM) to block FRs on the cell surface. The IGROV-1 tumor cells were incubated for 1 h at 4° C. on a shaker, followed by removal of the supernatants and rinsing the cells twice with PBS. After lysis of the cells with NaOH solution (1 M, aq., 500 μL), the samples were counted for activity in a γ-counter (Wallac Wizard 1480, Perkin Elmer). The counts per minute (cpm) of the specific binding (determined by subtracting the cpm of the unspecific binding from the cpm of total binding), were plotted against the molar concentration of the added folate radioconjugates of the disclosure. The nonlinear regression analysis for determination of the K_{D} value was performed using GraphPad Prism software (version 8). The results were expressed as the average of the K_{D} values of 3 experiments performed in triplicate and the respective 95% confidence interval. All folate radioconjugates showed FR-binding affinities in the low nanomolar range (K_{D}=1.4-5.6 nM) (Table 6).

TABLE 6
K_{D} values determined for each folate
radioconjugate of the disclosure.

| | K_{D} | 95% | |
|---|---|---|---|
| | Value | Confidence | |
| Radioconjugate | [nM] | interval | n |
| [¹⁷⁷Lu]Lu-6R-RedFol-24 | 5.6 | 3.3-8.8 | 3 |
| [¹⁷⁷Lu]Lu-6S-RedFol-24 | 4.6 | 3.2-6.4 | 3 |

### Example 8: Biodistribution Studies

Five week-old female, athymic nude mice (CD-1 Foxn1^{nu}) were purchased from Charles River Laboratories (Sulzfeld, Germany) and fed ad libitum with a folate-deficient rodent chow (ssniff Spezialdiaten GmbH, Germany). After an acclimatization period of at least 7 days, the mice were subcutaneously inoculated with KB tumor cells (5×10⁶ cells in 100 μL of PBS) on the shoulder. Approximately 2 weeks later, biodistribution studies were performed after intravenous administration of the folate radioconjugates of the disclosure (3 MBq, 0.5 nmol, 100 μL PBS containing 0.05% BSA) using n=3-4 mice per timepoint. At 1 h, 4 h and 24 h post injection (p.i.), the mice were sacrificed and selected tissues and organs were collected, weighed and counted for activity using a gamma-counter (PerkinElmer Wallac Wizard 1480). The results were reported as the percentage of the injected activity per gram of tissue mass (% IA/g) using counts of a defined volume of the original injection solution measured at the same time to obtain decay-corrected data.

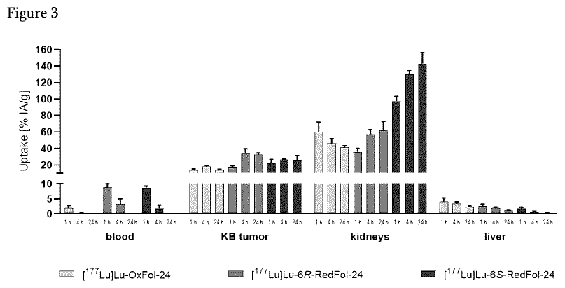

Biodistribution data (in particular the uptake in KB tumors and various other tissues as well as retention in the blood) were obtained at variable time points after injection of the folate radioconjugates of the disclosure (FIG. 3).

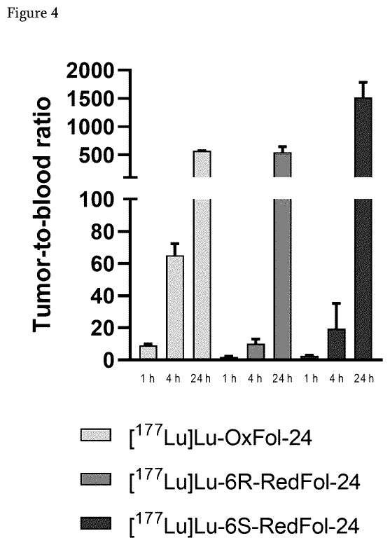

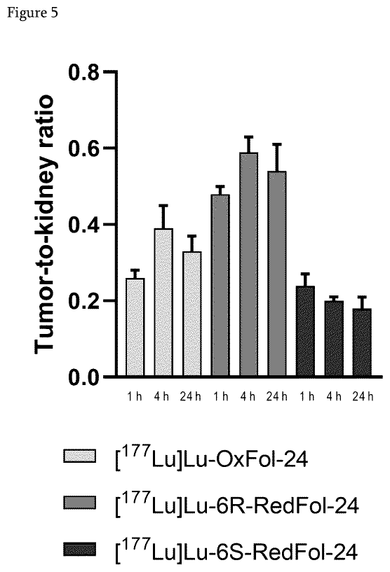

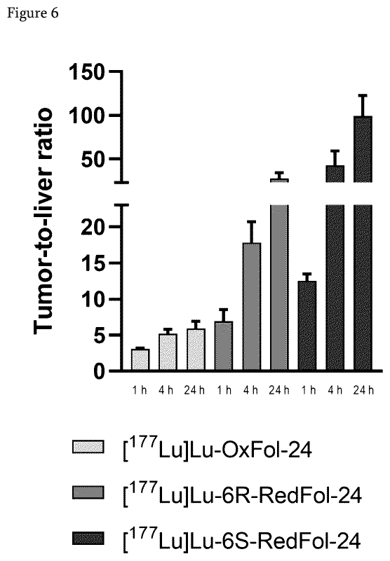

The tumor-to-blood, tumor-to-kidney, and tumor-to-liver ratios observed for the conjugates of the disclosure are shown in FIG. 4, FIG. 5, and FIG. 6, respectively.

### Example 9: SPECT/CT Imaging Studies

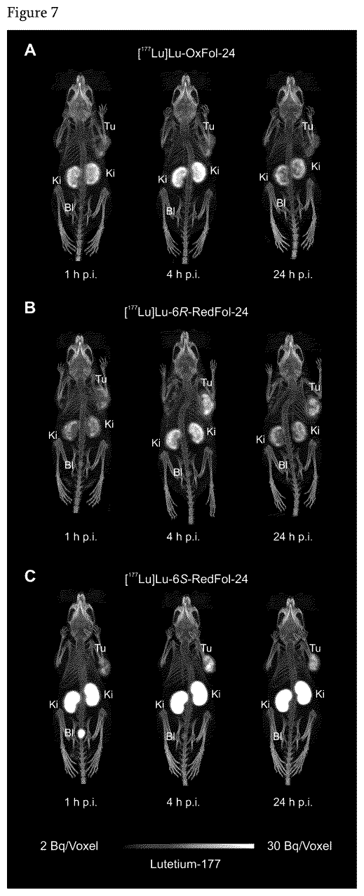

SPECT/CT imaging studies were performed to investigate the whole body distribution of folate radioconjugates of the disclosure in KB tumor-bearing mice (FIG. 7). SPECT/CT experiments were performed approximately 2 weeks after tumor cell inoculation when the tumor size reached a volume of ca. 300 mm³. Mice were injected with the folate radioconjugates of the disclosure (25 MBq, 0.5 nmol, 100 μL, diluted in PBS containing 0.05% BSA) and scanned at 1 h, 4 h, 24 h and 48 h p.i. Imaging studies were performed using a four-head, multiplexing, multipinhole small-animal SPECT camera (NanoSPECT/CT™, Mediso Medical Imaging Systems, Budapest, Hungary) as previously reported. Each head was outfitted with a tungsten-based aperture of nine 1.4 mm-diameter pinholes and a thickness of 10 mm. CT scans of ca. 7.5 min duration were followed by SPECT scans of ca. 40 min. The images were acquired using Nucline Software (version 1.02, Mediso Ltd., Budapest, Hungary). The real-time CT reconstruction used a cone-beam filtered backprojection. The reconstruction of SPECT data was performed with HiSPECT software (version 1.4.3049, Scivis GmbH, Gottingen, Germany) using γ-energies of 56.1 keV (±10%), 112.9 keV (±10%) and 208.4 keV (±10%) for lutetium-177. Images were prepared using VivoQuant post-processing software (version 3.5, inviCRO Imaging Services and Software, Boston, U.S.). A Gauss post-reconstruction filter (FWHM=1.0 mm) was applied and the scale of activity was set as indicated on the images (minimum value=2 Bq/voxel to maximum value=30 Bq/voxel).

FIG. 7 is showing SPECT/CT images as maximum intensity projections (MIPs) of KB tumor-bearing mice 1 h, 4 h, and 24 h after injection of the ¹⁷⁷Lu-folate radioconjugates (25 MBq; 0.5 nmol per mouse). (A) SPECT/CT scans of 6R-5-MTHF-based radioconjugates; (B) SPECT/CT scans of 6S-5-MTHF-based radioconjugates; Tu=KB tumor; Ki=kidney; H=heart.

1. A compound of formula I, Ia or Ib or a pharmaceutically acceptable salt thereof 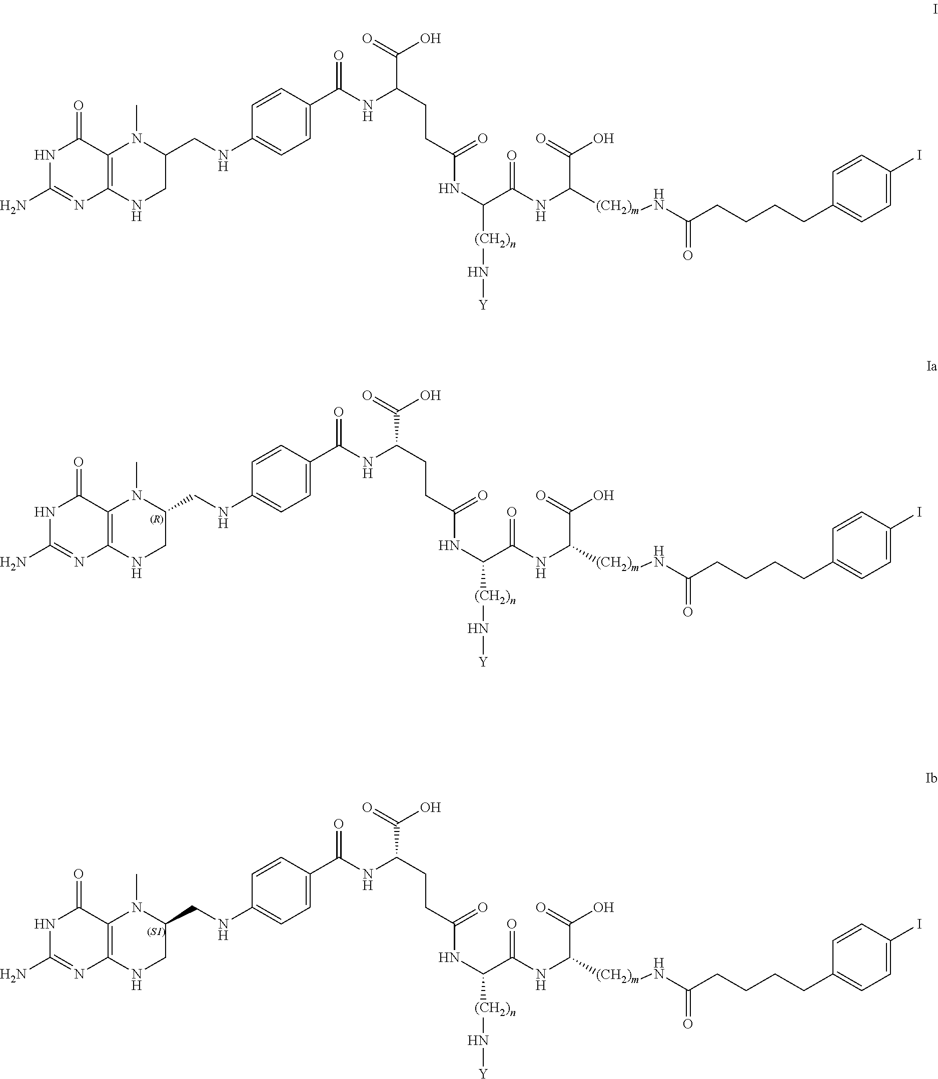
wherein
Y is a radiometal chelator optionally coordinating a radiometal M,
n is 1 to 8, and
m is 1 to 8.

2. A compound according to claim 1, wherein the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, and MECAM.

3. A compound according to claim 1, wherein the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

4. A compound according t wherein n is 2, 3, 4, 5 or 6, preferably 4, and/or m is 2, 3, 4, 5 or 6, preferably 4.

5. (canceled)

6. A compound according to claim 1, wherein n is 4 and m is 4.

7. A compound according to claim 1 having formula II, IIa or IIb 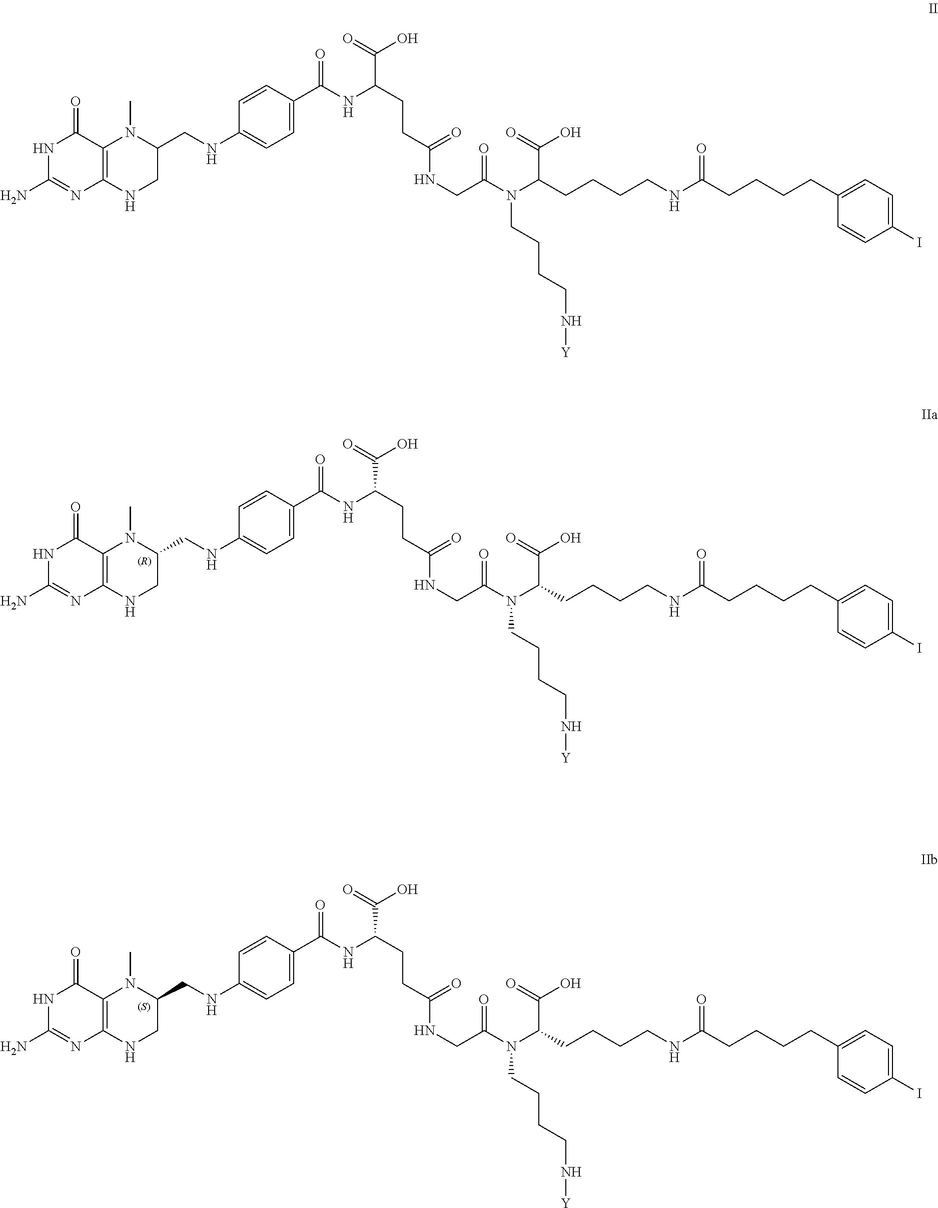
wherein Y is a radiometal chelator optionally coordinating a radiometal M.

8. A compound according to claim 7, wherein the radiometal chelator is selected from linear or macrocyclic polyaminocarboxylates, such as DTPA, DOTA (and derivatives thereof, such as p-SCN-DOTA, maleimido-DOTA, DOTA-NHS-ester), DFO, DFO*, DO3A, HP-DO3A, AAZTA, EDTA, TETA, EHPG, HBED, NOTA (and derivatives such as p-SCN-NOTA), DOTAGA, DOTMA, TETMA, PDTA, TTHA, LICAM, and MECAM.

9. A compound according to claim 7, wherein the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

10. A compound according to claim 1 having formula III, IIIa or IIIb optionally coordinated radiometal M

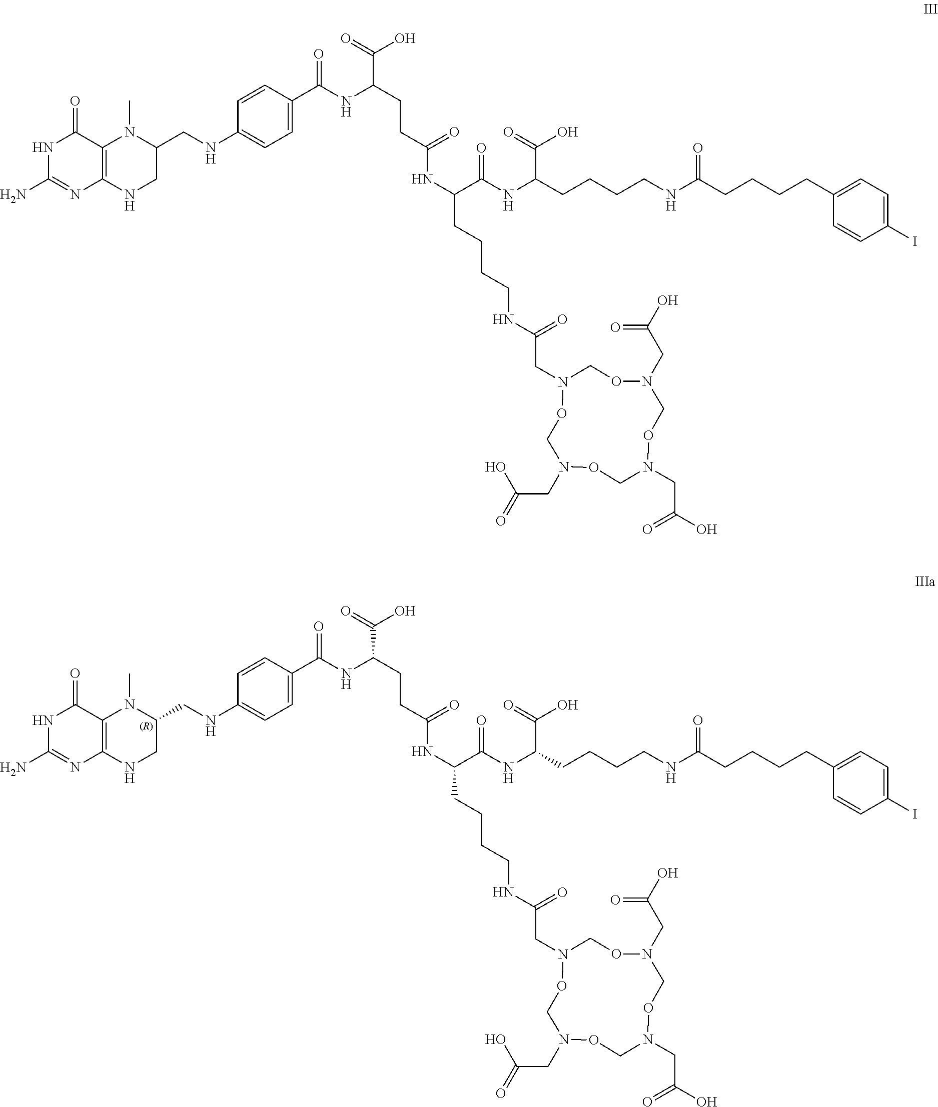

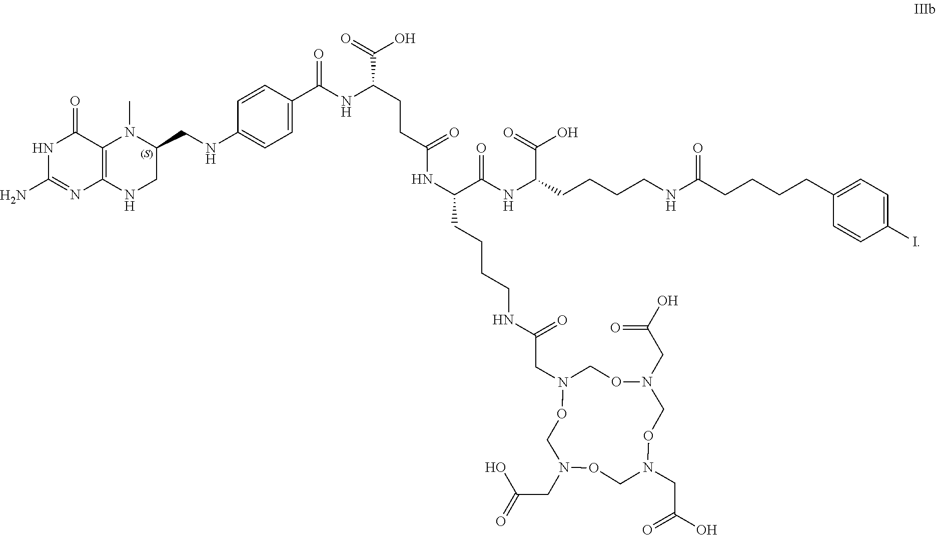

11. A compound according to claim 10, wherein the optionally coordinated radiometal M is selected from ⁵¹Cr, ⁶⁷Ga, ⁶⁸Ga, ⁴³Sc, ⁴⁴Sc, ⁴⁷Sc, ¹⁶⁷Tm, ¹⁴¹Ce, ¹¹¹In, ¹⁶⁸Yb, ¹⁷⁵Yb, ¹⁴⁰La, ⁸⁹Zr, ⁹⁰Y, ⁸⁸Y, ¹⁵³Sm, ¹⁶⁶Ho, ⁵²Mn, ¹⁶⁵Dy, ¹⁶⁶Dy, ⁶¹Cu, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ²¹²Pb, ^{117m}Sn, ¹⁴⁹ Pm, ¹⁶¹Tb, ¹⁴⁹Tb, ¹⁵²Tb, ¹⁵⁵Tb, ^{99m}Tc, ¹⁶⁵Er, ¹⁶⁹Er, ¹⁷²Yb, ¹⁶⁵Tm, ¹⁷⁷Lu, ²²⁵Ac, ¹⁹⁸Au, ¹⁹⁹Au, and ²²⁷Th.

12. Pharmaceutical composition comprising at least one compound according to claim 1 and a pharmaceutically acceptable carrier.

13. (canceled)

14. (canceled)

15. (canceled)

16. (canceled)

17. (canceled)

18. Method for radionuclide therapy comprising the steps of administering to the subject in need thereof at least one compound according to claim 1 in therapeutically effective amounts, localizing the at least one compound in a tissue to be treated, and subjecting the tissue to radiation to achieve therapeutic effect.

19. Method for therapeutic planning and/or monitoring and/or treatment of a tumor comprising the steps of (i) administering at least one compound according to claim 1 in diagnostically effective amounts to obtain a diagnostic image, and (ii) administering at least one further compound according to claim 1 in therapeutically effective amounts for tumor treatment, wherein optionally the diagnostic image obtained from the at least one compound administered in diagnostically effective amounts in step (i) is used to determine the therapeutically effective amount of the at least one further compound administered in step (ii) for treatment.

20. Compound according to claim 19 wherein the at least one compound of step (i) is a diagnostic compound and the at least one further compound of step (ii) is a radiotherapeutic compound.

21. (canceled)

22. Method for diagnostic imaging of a cell or population of cells expressing a folate-receptor, said method comprising the steps of administering at least one compound according to claim 1 in a diagnostically effective amount, and obtaining a diagnostic image of said cell or population of cells, wherein optionally the diagnostic imaging is performed of a cell or population of cells expressing a folate-receptor in vitro or in vivo.

23. (canceled)

24. Method for in vitro detection of a cell expressing the folate receptor in a tissue sample which includes contacting said tissue sample with a compound according to claim 1 in diagnostically effective amounts and for sufficient time and conditions to allow binding to occur and detecting such binding by PET imaging.

25. Method for diagnostic imaging or monitoring a subject comprising the steps of (i) administering at least one compound according to claim 1 in a diagnostically effective amount, and (ii) performing diagnostic imaging using PET by detecting a signal from said at least one compound.

26. Method for therapeutic planning of a treatment in a subject comprising the steps of (i) administering to a subject in need thereof at least one compound according to claim 1 in diagnostically effective amounts to obtain a diagnostic image, and (ii) determining the therapeutically effective amount of at least one further compound according to claim 1 to be administered for treatment.

27. Method for a theragnostic application comprising the steps of (i) administering to a subject in need thereof at least one compound according to claim 1 in a diagnostically effective amount, (ii) obtaining a diagnostic image of said at least one compound in the tissue to be treated, (iii) administering at least one further compound according to claim 1 in a therapeutically effective amount and (iv) subjecting the tissue to radiation, to achieve a therapeutic effect, wherein optionally the diagnostic image obtained in step (ii) is used to determine the therapeutically effective amount of the at least one further compound administered is step (iii) for treatment.

28. Method according to claim 27 wherein the at least one compound of step (i) is a diagnostic imaging compound and the at least one further compound (of step (ii) is a radiotherapeutic compound.

29. (canceled)

30. (canceled)
